(19) United States
(12) Patent Application Publication
(10) Pub. No.: US 2009/0264911 A1
(43) Pub. Date: Oct. 22, 2009

(54) SINGLE-USE DISPOSABLE LANCING APPARATUS AND METHODS

(76) Inventor: Stanley I. Kim, Upland, CA (US)

Correspondence Address: MARLIN J MESSICK, 1553 FOREST AVENUE, SARALAND, AL, 36571, US

(21) Appl. No.: 12/424,396

(22) Filed: Apr. 15, 2009

Related U.S. Application Data
(60) Provisional application No. 61/124,282, filed on Apr. 16, 2008.
(60) Provisional application No. 61/127,155, filed on May 10, 2008.

(51) Int. Cl. A61B 17/32

(52) U.S. Cl. 606/182; 606/181

(57) ABSTRACT

A single-use disposable lancing apparatus configured to extend and rapidly retract a lancet needle for reduced pain in taking body fluid samples, such as blood samples from skin. Embodiments include use of differential pressure, pneumatics, partial vacuum, compressed gas or fluid to actuate the lancet needle. Variations include the use of relatively increased pressure in an anterior chamber, relatively decreased pressure in a posterior chamber, or both. Additional optional springs may be used to supplement the pressure driven actuation of the lancing apparatus.

### CROSS-REFERENCE TO RELATED APPLICATIONS

This application claims the benefit of priority from U.S. Provisional No. 61/124,282 filed Apr. 16, 2008 and U.S. Provisional No. 61/127,155 filed May 10, 2008, which are incorporated in their entirety by reference, herein.

### BACKGROUND OF THE INVENTION

1. Field of the Invention

Various embodiments of the present inventions relate to single use disposable lancing devices and related methods.

2. Description of the Related Art

As more laboratory tests can be done at home or doctor's office rather than at the laboratory facility, the need of safe and comfortable disposable lancet is increasing. For example, tests such as measuring blood glucose, Protime (the prothrombin time) or cholesterol can be done at home or doctor's office.

For example, finger prick blood tests for glucose, cholesterol, anemia and coagulation are commonly performed at home or doctor's office. The results of the tests will be available immediately. Otherwise, the patient has to go to a laboratory facility and undergo procedures, such as a phlebotomy that involves a skin puncture of the arm by a 21 gauge thick needle, and return to the doctor's office for the test results as the laboratory will not give the test results to the patient directly. Therefore, finger prick blood tests can save time, expense and pain.

Currently, there are two primary types of lancets being used at home or office. The first is a disposable lancet having a needle mounted on a plastic lancet body that is used in conjunction with a reusable lancing device. These are widely used at home by diabetic patients who puncture the finger skin to obtain a drop of blood to measure the glucose level. However, this type of lancing procedure requires insertion and removal of the lancet in and out of the reusable lancing device by hand, which can increase the risk of accidental needle stick.

The second primary type of lancet being used at home or the office relates to certain types of disposable lancing devices consisting of a lancet and a housing having an actuation mechanism.

Certain existing lancets with actuation mechanisms tend to be safer as handling the lancet needle is not necessary as the disposable lancing device already has a lancet assembly and certain spring propelled mechanisms in the housing. Once the lancing has been done, the lancing device is simply discarded. If the lancet needle can be retracted after use, the risk of accidental needle stick is almost nil.

However, existing disposable lancing devices can be expensive to manufacture because they have several components such as the lancet, the spring, the actuator and the housing, and it frequently needs to be assembled manually. In addition, because the disposable lancing device can not be reused after initial use, the lancet needle is usually thicker and longer to ensure adequate bleeding by single lancing procedure. This can result in increased puncture pain.

### SUMMARY OF THE INVENTION

Accordingly, there is a need for a lancet system that rapidly deploys and retracts a lancet needle in and/or out of skin to reduce pain associated with the sampling or withdrawal of bodily fluid such as blood from skin. There is provided in accordance with one embodiment of the present invention a lancing apparatus 11, 60 configured to reduce pain in taking body fluid samples from the skin, the lancing apparatus comprising a housing 12 and an actuation assembly 13. In one embodiment the housing 12 has an inner space 49. In one embodiment the actuation assembly 13 includes a lancet needle 15, a divider 18 and an actuator 21. In one embodiment the lancet needle 15 has a retracted needle configuration 55 and an extended configuration 53. The retracted needle configuration 55 encloses the lancet needle 15 within the housing 12. The extended configuration 53 at least partially extends the lancet needle 15 outside the housing 12. The lancet needle 15 is operably connected to and extends distally from the divider 18. In one embodiment the divider 18 is operably connected to and distal to the actuator 21. The divider 18 has a divider front surface 50 and a divider back surface 51. In one embodiment the divider 18 is axially moveable within the inner space 49. In one embodiment the actuator 21 is configured for actuating the lancet needle 15 from the retracted needle configuration 55 to the extended configuration 53. In one embodiment at least one of the divider front surface 50 and the divider back surface 51 is fluidly connected to a substantially sealed chamber 57 within the inner space 49. In one embodiment the substantially sealed chamber 57 is configured for using a relative differential pressure (not shown in the Figures) to actuate the lancet needle 15 from the extended configuration 56 to the retracted needle configuration 55.

In various embodiments, the substantially sealed chamber 57 is a posterior chamber 48 with a relatively lower differential pressure. In various embodiments, the posterior chamber 48 fluidly connected to the divider back surface 51. In various embodiments, the substantially sealed chamber 57 is an anterior chamber 47 with a relatively higher differential pressure. In various embodiments, the anterior chamber 47 fluidly connected to the divider front surface 50. In various embodiments, the substantially sealed chamber 57 is a posterior chamber 48 is fluidly connected to the divider back surface 51. The posterior chamber 48 has a relatively lower differential pressure than an anterior chamber 47 that is fluidly connected to the divider front surface 50. In one embodiment the lancing apparatus 11, 60 also includes at least one repelling spring 19 operably connected to the divider front surface 50. In one embodiment the lancing apparatus 11, 60 also includes at least one actuation spring 20 operably connected to the divider back surface 51.

There is provided in accordance with one embodiment of the present invention a differential pressure actuated lancing apparatus 11, 60 that includes a housing and an actuation assembly 13. In one embodiment the housing 12 has an inner space 49, the inner space 49 having a posterior chamber 48 and an anterior chamber 47. In one embodiment the actuation assembly 13 includes a lancet needle 15 and a divider 18. In one embodiment the lancet needle 15 has a retracted needle configuration 55 and an extended configuration 56. In one embodiment the retracted needle configuration 55 encloses the lancet needle 15 within the housing 12. In one embodiment the extended configuration 56 at least partially extends the lancet needle 15 outside the housing 12. In one embodiment the lancet needle 15 is operably connected to and extending distally from the divider 18. In one embodiment the divider 18 has a divider front surface 50 in fluid communication with the anterior chamber 47 In one embodiment the divider 18 has a divider back surface 51 in fluid communication with the posterior chamber 48. In one embodiment the divider 18 axially moveable within the inner space 49. In one embodiment the anterior chamber 47 has a relatively higher differential pressure than the posterior chamber 48 in the extended configuration 56. In one embodiment the differential pressure actuates the divider 18 proximally toward the anterior chamber 47.

In one embodiment the differential pressure actuated lancing apparatus 11, 60 also includes at least one repelling spring 19 operably connected to the divider front surface 50 to assist in retracting the lancet needle 15 in to the housing 12. In one embodiment the differential pressure actuated lancing apparatus 11, 60 also includes at least one actuation spring 20 operably connected to the divider back surface 51. In one embodiment the differential pressure actuated lancing apparatus 11, 60 also includes at least one actuation spring 20 configured to assist in exposing the lancet needle 15 out of the housing 12. In one embodiment the differential pressure actuated lancing apparatus 11, 60 also includes at least one actuation spring 20 configured to assist in retracting the lancet needle 15 in to the housing 12. In one embodiment the differential pressure actuated lancing apparatus 11, 60 also includes an actuator 21 mechanically connected to the divider 18 for releasing the divider 18 from a locked retracted needle configuration 55.

There is provided in accordance with one embodiment of the present invention a method of actuating a lancing apparatus 11, 60 including the steps of actuating an actuator 21 and retracting a lancet needle 15. The step of actuating an actuator 21 includes an actuator 21 connected to a lancet needle 15 and a divider 18, the actuator 21 releasing the lancet needle 15 from a retracted needle configuration 55 to an extended configuration 56. In one embodiment the retracted needle configuration 55 encloses the lancet needle 15 within a housing 12. In one embodiment the extended configuration 56 at least partially extends the lancet needle 15 outside the housing 12. In one embodiment the divider 18 is operably connected to and distal to the actuator 21, the divider 18 having a divider front surface 50 and a divider back surface 51. In one embodiment the divider 18 axially moveable within the housing 12. In one embodiment the step of retracting the lancet needle 15 from the extended configuration 56 to the retracted needle configuration 55 uses a relatively lower differential pressure in a posterior chamber 48 fluidly connected to the divider back surface 51 relative to an anterior chamber 47 fluidly connected to the divider front surface 50.

In one embodiment the method of actuating a lancing apparatus 11, 60 also includes advancing the lancet needle 15 out of the housing 12 with at least one actuation spring 20 operably connected to the divider back surface 51. In one embodiment the method of actuating a lancing apparatus 11, 60 also includes retracting the lancet needle 15 in to the housing 12 with at least one actuation spring 20 operably connected to the divider back surface 51. In one embodiment the method of actuating a lancing apparatus 11, 60 also includes retracting the lancet needle 15 in to the housing 12 with at least one repelling spring 19 operably connected to the divider front surface 50.

### BRIEF DESCRIPTION OF THE DRAWINGS

These and other features, embodiments, and advantages of the present invention will now be described in connection with preferred embodiments of the invention, in reference to the accompanying drawings. The illustrated embodiments, however, are merely examples and are not intended to limit the invention.

FIG. 1 is a schematic perspective posterior upper view of the disposable lancing device having the actuation assembly shown as dotted lines, according to one embodiment of the present invention.

FIG. 2 is a schematic perspective posterior upper view of the housing of the disposable lancing device of FIG. 1.

FIG. 3 is a schematic perspective posterior upper view of the actuation assembly of the disposable lancing device of FIG. 1.

FIG. 4 is the schematic front view of the disposable lancing device of FIG. 1.

FIG. 5 is the schematic back view of the disposable lancing device of FIG. 1.

FIG. 6 is the schematic front view of the actuation assembly of FIG. 3.

FIG. 7 is the schematic partial sectional lateral view without the side wall of the disposable lancing device of FIG. 1, without the front barrel and the anterior wall of the main case.

FIG. 8 is a schematic cross sectional view at the line A of FIG. 7.

FIG. 9 is a schematic cross sectional view at the line B of FIG. 7.

FIGS. 10A and 10B are schematic sectional top views showing various steps in the actuation of the lancet of the disposable lancing device of FIG. 1.

FIG. 11 is a schematic perspective posterior upper view of a single use disposable lancing device according to one embodiment of the present invention.

FIG. 12 is a perspective posterior upper view of the housing of the single use disposable lancing device of FIG. 11.

FIG. 13 is a schematic perspective posterior upper view of the actuation assembly of the single use disposable lancing device of FIG. 11.

FIG. 14 is a schematic front view of the lancing device of FIG. 11.

FIG. 15 is a schematic back view of the lancing device of FIG. 11.

FIG. 16 is a partial sectional side view of the lancing device of FIG. 11.

FIG. 17 is a schematic cross section view at the line A of FIG. 16.

FIG. 18 is a schematic cross section view at the line B of FIG. 16.

Throughout the figures, the same reference numerals and characters, unless otherwise stated, are used to denote like features, elements, components or portions of the illustrated embodiments. In certain instances, similar names may be used to describe similar components with different reference numerals which have certain common or similar features. Moreover, while the subject invention will now be described in detail with reference to the figures, it is done so in connection with the illustrative embodiments. It is intended that changes and modifications can be made to the described embodiments without departing from the true scope and spirit of the subject invention as defined by the appended claims. In the following detailed description of some embodiments of the present invention will be given with reference to the drawings. However, the invention is not to be considered as restricted to these embodiments. In addition, the signs in the drawing are not restricted to be used only as marked.

### DETAILED DESCRIPTION

As should be understood in view of the following detailed description, this application is primarily directed to improvements to apparatuses, systems and methods associated with lancets for taking body fluid samples, such as blood. To facilitate the understanding of the reference numbers used in the description below with respect to the Figures, the following listing of reference numbers for certain embodiments is provided:
11. A disposable lancing device
12. Housing
13. Actuation assembly
14. Needle protector
15. Lancet needle
16. Pedestal for the lancet needle
17. Lancet body
18. Divider
19. Repelling spring
20. Actuation spring
21. Actuator
22. Front barrel
23. Main case
24. Raised side rail
25. Concave groove on the outer side wall
26. Concave groove on the upper and lower wall
27. Half column stopper
28. Front wall of the case
29. Side wall of the housing
30. Posterior opening of the main case
31. Recess
32. Flange
33. End cap
34. Tip of the front barrel
35. Base plane of the actuator
36. Round wedge of the divider wing
37. Head of the divider
38. Neck of the divider
39. Front exit opening of the lancing device
40. Side wall
41. Upper wall
42. Lower wall
43. Upper wall groove
44. Lower wall groove
45. Open recess
46. Convex lateral edge of the divider
47. Anterior chamber
48. Posterior chamber
49. Inner space of housing
50. Divider front surface
51. Divider back surface
52. First configuration
53. Second configuration
54. Third configuration
55. Retracted needle configuration
56. Extended needle configuration
57. Substantially sealed chamber
60. A disposable lancing device

### OBJECTIVES AND ADVANTAGES

In one embodiment of the present invention, an objective of the present invention to produce a high quality, safe and less expensive disposable lancing device. Certain objectives and advantages of embodiments of the present invention include: 1. Minimal puncture pain; 2. No risk of needle stick injury after use; 3. Simple structure and least number of components; 4. Simple and fast automatic assembling with minimal cost of production of the highest quality product; and 5. Reliable blood sampling. In order to achieve above goals, novel and innovative mechanisms and features are disclosed in various embodiments of the present invention as described further below.

1. Minimal Puncture Pain.

To minimize puncture pain, in various embodiments of a disposable lancing device 11, 60, at least three different types of mechanisms and features can be used alone or in combination to retract the lancet needle 15 from the skin as quickly and rapidly possible once the lancet needle 15 has punctured tissue, such as skin. The shorter the duration of time that the lancet needle 15 stays inside the skin, the less the irritation of pain nerve fibers and puncture pain. Each of the features, alone or in combination, help to retract a lancet needle 15 quickly, thus lessening puncture pain. In one embodiment the lancet needle 15 has a first configuration 52 enclosed within the housing 12. In one embodiment the first configuration 52 completely encloses the lancet needle 15 in the housing 12. In one embodiment the first configuration 52 holds the lancet needle 15 in a releasably locked position. In one embodiment the lancet needle 15 has a second configuration 53 extending at least partially outside the housing 12. In one embodiment the second configuration 53 is attained by releasing the lock or trigger or actuation of the lancet needle 15 from the first configuration 52. In one embodiment the lancet needle 15 has a third configuration 54 enclosed within the housing 12. In one embodiment the third configuration 54, with the lancet needle 15 enclosed within the housing 12, is similar to the first configuration 52 with the lancet needle 15 released from its locked position. In one embodiment the first configuration 52 corresponds to the lancet needle 15 configuration before firing the device 11, 60 and the third configuration 54 corresponds to the lancet needle 15 configuration after firing the device 11, 60 and having the lancet needle 15 temporarily extend to the second configuration 53. In various embodiments, the first configuration 52 and the third configuration 54 are retracted needle configurations 55. In one embodiment the second configuration 53 is an extended needle configuration 56.

In various embodiments, a disposable lancing device 11, 60 comprises a substantially sealed chamber 57 within the housing 12. As used herein, “substantially sealed chamber” can be used to describe a completely sealed chamber, a partially sealed chamber, or a chamber with a moveable wall that does not necessarily perfectly seal a chamber, but can temporarily maintain a partial differential in pressure when the moveable wall is moved or actuated by hand or the release of a trigger or lock with springs. In one embodiment, a disposable lancing device 11, 60 comprises a substantially sealed chamber 57 within the housing 12 configured to use a differential pressure to actuate the lancet needle 15 between a retracted needle configuration 55 and an extended needle configuration 56. In one embodiment, a disposable lancing device 11, 60 comprises a substantially sealed chamber 57 within the housing 12 configured to use a differential pressure to actuate the lancet needle 15 between extended needle configuration 56 and a retracted needle configuration 55. For example, in one embodiment the substantially sealed chamber 57 retracts the lancet needle 15 proximally quickly by a temporary difference in air pressure. In various embodiments the quick proximal retraction of the lancet needle can be driven by relatively higher pressure in an anterior chamber 47, or by relatively lower pressure in a posterior chamber 48, or both. In one embodiment, when the lancing device is about to be fired, the anterior chamber 47, which includes the inner space of the housing in front of a divider, pedestal, piston or membrane attached to the needle, is a substantially closed space with the front exit opening of the housing (for the needle) completely sealed as it is in close contact with the skin. In one embodiment the posterior chamber 48 includes the inner space of the housing behind, or posterior, or proximal to a divider, pedestal, piston or membrane attached to the needle and the end of the housing which can be closed by an end cap. In one embodiment, a divider of the lancet assembly occupies most of the cross sectional area of the closed housing space and divides the space into two compartments; the anterior chamber or front compartment in its front and the posterior chamber, rear compartment, or posterior compartment in its back space. In one embodiment additional springs may be used to assist in the movement of the needle. In one embodiment, as the needle moves forward forcefully by the actuation spring inside the closed space such as a piston in the cylinder, the air in the front compartment of the housing is compressed, while the posterior compartment creates a negative air pressure vacuum. The compressed air pressure of the front compartment and negative air pressure in the posterior compartment reach the highest level when the lancet punctures the skin, ready to push the divider back. Therefore, the lancet assembly in connection with the divider will be immediately repelled backward in the housing until the air pressure between the two compartments is balanced. Therefore, the lancet needle stays in the skin for extremely short period of time, reducing irritation of the pain nerve.

In various embodiments, the use of pressure to drive the needle can involve the use of gas, fluid, air, pneumatics, or other pressure delivery materials. Unlike other vacuum related lancets of the art which use a partial vacuum in an anterior portion of those lancets to suck blood or fluid out of the skin, certain embodiments of the present invention use a higher pressure in an anterior chamber to drive the needle backward to retract the needle, or a lower pressure in the posterior chamber to drive the needle backward to retract the needle, or both. Other lancets which use suction at the anterior portion of their lancets to draw blood out tend to use low pressure at the skin surface, and need to have some extended contact to draw the blood out.

In various embodiments, the substantially sealed chamber 57 is a partial seal. In one embodiment, there is no differentiation of the relative pressure in the anterior and posterior chamber before firing. In one embodiment, a temporary pressure gradient is generated while the lancet needle 15 is fired by the release of one or more actuation springs. In one embodiment, the main force generating the pressure gradient is driven by the release of a compressed actuation spring, such as when an actuator is activated, released, or pushed. In one embodiment, a partial or complete sealing is generated when the exit opening of the lancet needle is in close contact with the skin before firing the lancet.

In one embodiment, a disposable lancing device 11, 60 enables the lancet needle to retract proximally, or be repelled backward quickly with a repelling spring mounted on the anterior surface of the divider 18 that was compressed against the inner front wall of the housing when the actuation of the spring pushed the divider will repel the divider as the actuation spring lost the compression pressure after the skin puncture. This feature adds more repelling force to shorten the time duration of the lancet needle staying inside the skin, reducing puncture pain. In addition, the repelling spring keeps the divider and lancet assembly from moving forward once the divider has been repelled because it will stretch back to its original length. Therefore, it keeps the retracted lancet needle from moving toward the skin again, preventing needle injury after initial skin puncture.

In one embodiment, a disposable lancing device 11, 60 enables the lancet needle to retract proximally, or be repelled backward quickly with an over-stretched spring of actuation mechanism after firing will retract backward slightly once the lancet needles punctures the skin, thereby shortening the lancet needle time staying in the skin.

In various embodiments, any of these structures may be combined.

2. No Risk of Needle Stick Injury After Use.

In one embodiment, a disposable lancing device 11, 60 eliminates needle stick injury once the lancing has been done. This feature can also eliminate blood-borne disease transmission such as AIDS and hepatitis. In one embodiment the lancet needle retracts back into the housing and stays there once the puncturing has been done. In one embodiment the repelling spring re-stretches back to its original length after the skin puncture holds the weight of the divider and lancet assembly, preventing them from moving forward towards the skin. In one embodiment, once the lancing procedure is completed, the disposable lancing device can not be re-cocked for reuse, eliminating the risk of blood-borne disease transmission. In one embodiment, when the lancing device is fired, the neck of the divider breaks so that the lancet can not be re-cocked. In one embodiment, the actuator can not be pushed further forward to push the lancet assembly after initial firing. In one embodiment the actuator can not be pushed further forward to push the lancet assembly after initial firing because the base plane of the actuator attached into the end cap is not too flexible to be stretched further.

3. Simple Structure and Small Number of Components.

In one embodiment, a disposable lancing device 11, 60 comprises a divider 18 of the actuation assembly engaged with the raised rails on each side walls of the housing allow smooth forward motion of the lancet assembly, reducing puncture pain further more. In one embodiment, the divider 18 is butterfly-shaped. In a manner similar to a monorail train, the divider 18 with the lancet assembly moves smoothly forward as the round wedge of the butterfly wing of the divider slides over the raised rail on the inner side wall of the main case of the housing. This feature allows the stable and smooth forward movement of the divider and lancet assembly by eliminating undesirable irregular shaking, and minimizes puncture pain.

4. Simple and Fast Automatic Assembling with Minimal Cost of Production of the Highest Quality Product.

In one embodiment, a disposable lancing device 11, 60 is very simple to manufacture and assemble. In one embodiment, a disposable lancing device 11, 60 can be produced in one step automatic mechanical process, which in turn helps to improve quality of the product with fewer defects and to reduce the production cost. If a lancing device has too many components to be produced in separate processes, it will increase the risk of occurrence of defective parts as well as the cost of production. In one embodiment, a disposable lancing device 11, 60 has two primary components; a housing 12 and an actuation assembly 13. In one embodiment, the housing 12 is simply a hollow plastic case with some moldings. In one embodiment, the actuation assembly 13 is made as one unitary body consisting of a lancet assembly with a divider 18, an actuation spring 20, and an end cap with an actuator 21. In one embodiment, a unique design and configuration allows all the components of the actuation assembly 13 to be made as one body by single injection molding procedure.

In one embodiment, a method of assembling the disposable lancing device 11, 60 is extremely simple with just one step process of inserting the actuation assembly 13 into the housing 12. In one embodiment, the actuation assembly 13 is inserted into the housing 12 until the flanges of the end cap click into the receiving open recess on the walls of the housing case. In one embodiment, the actuation spring 20 will be compressed automatically during the insertion. In one embodiment, the assembling process can be done right after the injection molding of the housing 12 and actuation assembly 13 at the same facility.

In one embodiment, by modifying the butterfly wing of the actuation assembly 13 as well as the structure configuration of the housing 12 case, the disposable lancing device 11, 60 can be produced even at a lower cost because of the simpler molding process, while having many of the advantages of other embodiments. In one embodiment the lateral edges of a butterfly-shape divider are convex instead of concave. In one embodiment the lateral edges of the butterfly-shape divider of the actuation assembly of the prior art have concave features that are supposed to engage the side rails of the housing main case. This feature prevents a side shaking of the lancet assembly, and thus reducing puncture pain. However, in one embodiment the concave lateral edges may be more difficult for the molding procedure. By making one embodiment of the lateral edges convex, it is easier for the molding process, and the convex lateral edges engaged with the reciprocally concave side wall of the main case of the housing still can prevent the side motion and shaking of the moving lancet body. In one embodiment, in order to engage the convex lateral edges of the butterfly-shape divider of the actuation assembly, the side wall of the main case of the housing needs to be modifies reciprocally.

5. Reliable Blood Sampling.

In one embodiment, a disposable lancing device 11, 60 is a reliable blood sampling with minimal puncture pain, comprising a very thin and short lancet needle mounted on a pedestal. With help of the small pedestal, the lancet needle can be very short and thin. Because the pedestal comes out of the exit opening, and can function as a primary stopper for the needle puncturing the skin, the lancet needle penetrates the skin in its full length regardless of the condition of the skin. In one embodiment, a disposable lancing device 11, 60 comprises a length of the needle that is very short, ranging from 0.5-1.0 mm. In one embodiment, a disposable lancing device 11, 60 comprises a 0.5 mm needle mounted on the pedestal, and the depth of skin penetration is predictable to be 0.5 mm. In various embodiments, the disposable lancing device 11, 60 can have several different models depending on the size of lancet needle used.

The objects and advantages of the various embodiments of the lancing device 11, 60 as they relate to the embodiments of the structure of the lancing device 11, 60 described in further detail below.

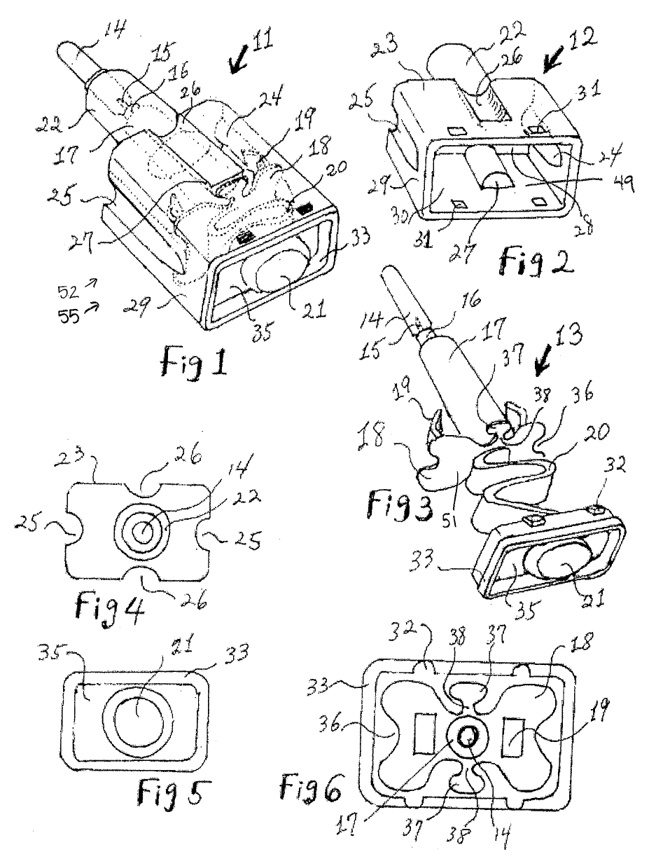

FIG. 1 shows one embodiment of a completely assembled disposable lancing device 11 comprised of the housing 12 and the actuation assembly 13. The actuation assembly 13 inside the housing 12 is shown as the dotted lines. FIG. 2 and FIG. 3 illustrate components of embodiments of the housing 12 and the actuation assembly 13. The disposable lancing device 11 is produced by simple automatic assembling of the two parts, the housing 12 and the actuation assembly 13, which are made by two separate single injection molding procedure. The uniquely designed actuation assembly 13 has one body composed of several components, and is produced in one injection molding procedure.

In one embodiment the housing 12 has an inner space 49. In one embodiment the actuation assembly 13 comprises a lancet needle 15 attached to a divider 18. In one embodiment the divider 18 has a divider front surface 50 and a divider back surface 51. In one embodiment the actuation assembly 13 also comprises a needle protector 14 in which the lancet needle 15 is embedded. In one embodiment the needle protector 14 is easily twisted off the lancet needle 15 before using the device 11. In one embodiment the lancet needle 15 is mounted on a pedestal 16 attached on a lancet body 17 that is again connected to the divider 18. In one embodiment the divider 18 divides the inner space of the housing 12 into the front compartment, or an anterior chamber 50, and a posterior compartment, or a posterior chamber 51. In one embodiment the divider front surface 50 faces the anterior chamber 47. In one embodiment the divider back surface 51 faces the posterior chamber 48.

In one embodiment the divider front surface 50 has one or more repelling spring 19 attached to it. In one embodiment, on each side of the divider front surface 50, attached are a pair of repelling springs 19. In one embodiment, on the divider back surface 51, attached are one or more actuation springs 20. In one embodiment the actuation spring 20 is mechanically linked or connected to an actuator 21. The base plane of the actuator 21 of rectangular shape suspends the actuator 21 to the end cap 33. In one embodiment the actuator 21 has a generally rectangular shape.

In one embodiment, all the components of the actuation assembly 13 can be made as one unitary body. In one embodiment the actuation assembly 13 is formed by one injection molding procedure because of the unique design and configuration. In one embodiment simple insertion of the actuation assembly 13 into the housing 12 completes the production of the disposable lancing device of the present invention.

In one embodiment the housing 12 of FIG. 2 comprises of the main case 23 and the front barrel 22 connected with a front wall 28. In one embodiment the main case 23 has the posterior opening 30 through which the actuation assembly 13 is inserted into the housing 12 for assembling, and on the front wall 28 is attached the front barrel 22. In one embodiment, on its outer walls, the main case 23 has two pairs of longitudinal grooves; the concave grooves with tailing ends on each side 25, and the concave grooves with abruptly cut ends on the upper and lower walls 26. In one embodiment the pair of the concave groove on the outer side walls 25 reciprocally become the pair of the raised side rails 24 on inner side walls.

In one embodiment the divider 18 engages with raised side rails 24 on each inner side walls of the housing case allow smooth forward motion of the lancet assembly, reducing puncture pain. This guide or rail system can be analogized to a smoothly moving train sliding on a monorail: the divider 18 along with the lancet body 17 moves smoothly forward as the round wedge of the butterfly wing 36 of the divider slides over the raised side rails 24 on the inner side wall of the main case 23.

In one embodiment the concave groove of the side wall 25 and its reciprocally raised side rail 24 are tapered off in the posterior end. The taping tails of the raised side rails 24 make it easy for the divider 18 to engage with the side rails 24 when the actuation assembly 13 is inserted into the housing 12 during the assembling process. In one embodiment the abruptly cut ends of the concave groove on the outer upper and lower walls 26 reciprocally become the half column stoppers 27 for the heads of the butterfly wing divider 37 on each inner side walls. The half column stoppers 27 on the upper and lower walls keep the actuation assembly 13 from moving forward until the lancet is fired. The stoppers 27 hold the head of the butterfly 37 resisting the pressure of the compressed actuation spring 20. In addition, this feature makes the assembling work extremely easy and simple.

As described in the objectives and advantages above, in one embodiment the assembling process involves simple one-step insertion of the actuation assembly 13 into the housing 12 until the flanges 32 of the end cap 33 click into the recess 31 of the housing case. In one embodiment the actuation spring 20 is automatically compressed during this insertion process as the half column stoppers 27 can hold the pressure of the compressing actuation spring 20 that is transmitted through the head of the butterfly 37 on it. In one embodiment, when the patient fires the lancing device by pressing the actuator 21, the narrow necks 38 of the divider 18 break. In one embodiment, when the patient fires the lancing device by pressing the actuator 21, the narrow necks 38 of the divider 18 is simply unlocked, toggled, or deflected. In one embodiment the actuation of the necks 38 of the divider 18 now allows the release of the compressed actuation spring 20, pushing forward the divider 18 along with the lancet body 17. Then the lancet needle 15 in front of the lancet body 17 punctures the skin. The forwardly moving divider 18 stops as it hits the inner side of the front wall of the case 28, and the lancet body 17 and the lancet needle 15 stop as well. By this time, the actuation spring 20 has lost the compression pressure, and the air pressure of the front compartment of the housing has been compressed by the forward moving divider 18 in the closed air space of the housing, and the repelling springs 19 on the divider 18 have been compressed against the inner surface of the front wall of the case 28. All is ready to repel the lancet needle 15 along with the lancet body 17 and the divider 8 backward. The lancet needle 15 will come out of the skin immediately and retracts back to the front barrel 22.

In one embodiment, once it is retracted, the needle can not go down or be exposed again to injure the skin because the re-stretched repelling spring 19 to its original length can hold the weight of the lancet needle 15, lancet body 17 and the divider 18.

In one embodiment the wall of the base plane of the actuator 35 is slightly thinner and flexible so that the actuator 21 can be pushed in for firing. In one embodiment the pressure generated by the pushing the actuator 21 overcomes the half column stopper 27 resisting the pressure of the actuation spring 20 by holding against the head of the butterfly 37, and actuates the narrow neck 38 of the butterfly. In various embodiments, the actuation of the parts or components can involve breaking, unlocking, toggling, deflecting, folding, or other types of actuation of the respective components.

FIG. 4 illustrates one embodiment of the lancing device of FIG. 1 showing the front barrel 22, the main housing case 23 and its front wall 28, and the concave grooves on the side walls 25 and on the upper and lower walls 26. FIG. 5 is a posterior view of an embodiment of the lancing device of FIG. 1 showing the end cap 33, the actuator 21 and the base plane of the actuator 35. FIG. 6 is a front view of an embodiment of the actuation assembly 13. In one embodiment the divider 18 has a pair of the head 37 and the neck 38, and its wing has the round wedge 36 on each side that engages the raised side rail 24 on the side walls of the housing case. FIG. 7 is the lateral view of an embodiment of the lancing device of FIG. 1 without the side wall.

In one embodiment the actuation spring 20 is compressed before firing, while the repelling spring 27 is not. Before lancing, the needle protector 14 is twisted off the lancet needle 15. When the actuator 21 is pushed by the finger, it in turn pushes the base plane of the actuator 35, the compressed actuator spring 20, and the divider 18. The narrow neck of the butterfly 38, the weakest part of the divider 18 actuates as it can no longer bear the pressure against the half column stopper 27. In various embodiments, the actuation of the parts or components can involve breaking, unlocking, toggling, deflecting, folding, or other types of actuation of the respective components. Then the compressed actuation spring 20 is suddenly released, thus moving the lancet assembly and the divider 18 forward.

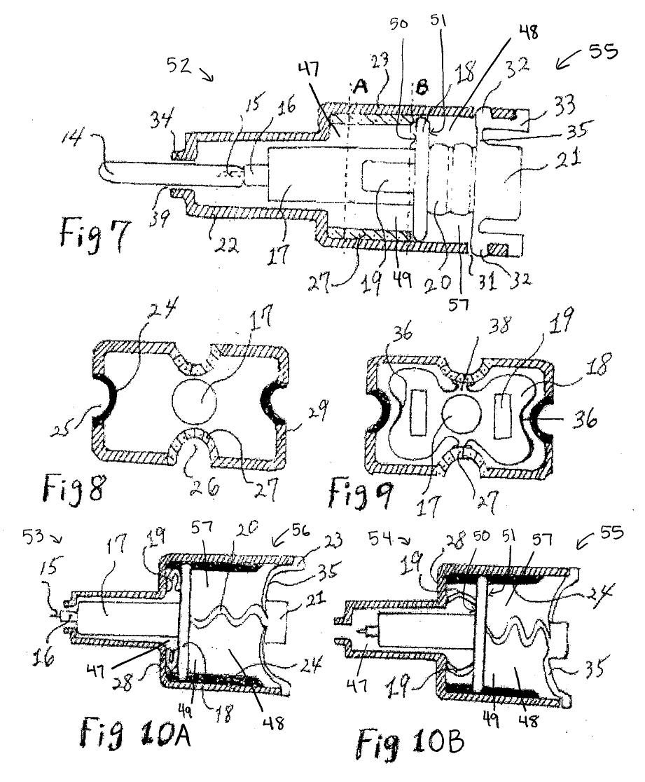

FIG. 8 is a cross section at the line A of FIG. 7, showing an embodiment of the lancet body 19, the concave grooves on the outer side walls 25 and their reciprocally raised side rails 24 inside the house. It also illustrates the concave grooves 26 on the upper and lower walls and their reciprocally convex half column stoppers 27. FIG. 9 shows the cross section at the line B of the FIG. 7, and illustrates an embodiment of how the pressure from the compressed actuation spring 20 is resisted. The half column stopper 27 holds the head of the butterfly bearing the pressure of the compressed spring 20. When more pressure is applied by pushing the actuator 21 for firing the lancing device, the narrow neck of the butterfly 38 can not hold the pressure any more and actuates, releasing the compressed actuation spring 20. In various embodiments, the actuation of the parts or components can involve breaking, unlocking, toggling, deflecting, folding, or other types of actuation of the respective components. This process moves the lancet assembly forward puncturing the skin.

FIGS. 10A and 10B are top views of an embodiment of the lancing device without the top wall in a firing mode. In one embodiment the actuator 21 is finger pressed, the actuation spring 20 released, and the divider 18 pushed along with the lancet body 17, the pedestal 16 and the lancet needle 15, puncturing the skin. The releasing pressure from the actuation spring 20 compresses the repelling springs 19 against the inner front wall of the housing 28. By then the actuation spring 20 looses the compression pressure, and the compressed repelling springs 19 easily repel the divider backward, retracting the lancet needle back into the front barrel 22. The retracted lancet needle 15 can not cause any needle stick injury. When the compressed spring 20 is released, it over-stretched initially, which causes slight retraction bi itself, further helping the retraction of lancet needle out of the skin after puncture.

In one embodiment, another novel mechanism of this present invention involves the air pressure helping the lancet needle retraction after puncturing the skin. In one embodiment the inner space of the housing 12 can be divided into two compartments by the divider 18; the anterior chamber 47, or a front compartment, and the posterior chamber 48, or a posterior compartment. In one embodiment the inside of the housing 12 is substantially completely closed space when the lancing device is ready to be fired as the exit opening 39 is sealed by the skin because of the tip of the front barrel 34 pressing the skin.

In one embodiment, when the lancing device is fired, the divider 18 moves forward by the forceful push of the actuation spring 20, creating positive air pressure in the front compartment and negative air pressure in the posterior compartment. The shape of the divider 18 occupies most of the inner cross sectional area of the housing functions as a septum in a closed space compartment, prohibiting the free air flow. By the time when the lancet needle 15 punctures the skin, the front compartment of the housing has rather high air pressure while the posterior compartment developed negative vacuum pressure. Then the positive pressure in the front compartment and the negative pressure in the posterior compartment force the divider 18 to move backward until the air pressure between the compartments becomes balanced. The lancet needle will be immediately retracts back because of the mechanism, and the patient feels only soft touch during the lancing procedure.

In one embodiment the divider completely fills the cross sectional area of the inner housing case and the air pressure can be adjusted. For example, when the divider 18 completely occupies the inner space like a syringe plunger in the barrel, the air pressure difference between the front and posterior compartment of the housing can be significant. The actuator 21 can not be pushed further forward because of the resisting base plane of the actuator 35 attached to the end cap 23. In one embodiment the base plane of the actuator 35 has a slightly thinner wall to be slightly flexible. In one embodiment it is not made as flexible as rubber.

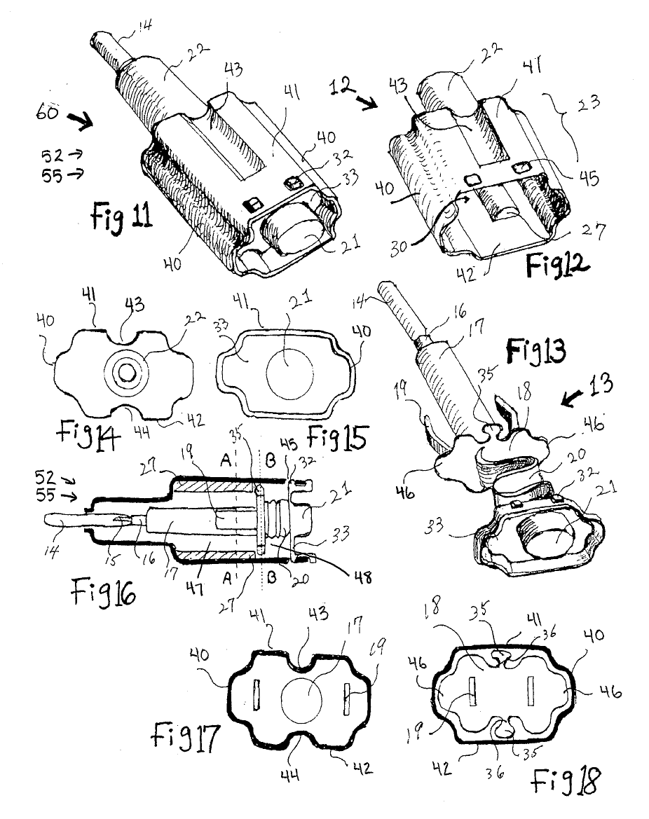

FIG. 11 shows one embodiment of a completely assembled disposable lancing device 60 comprised of two parts; the housing 12 and the actuation assembly 13. In various embodiments, the disposable lancing device 60 is the same or similar to the disposable lancing device 11 except as otherwise noted. Aspects of disposable lancing device 11 and disposable lancing device 60 can be combined. FIG. 12 and FIG. 13 illustrate the components of the housing 12 and the actuation assembly 13. In one embodiment the disposable lancing device 60 is produced by simple automatic assembling of the two parts, the housing 12 and the actuation assembly 13, which are made by two separate single injection molding procedure. The uniquely designed actuation assembly 13 has a single body composed of several components, and is produced in one injection molding procedure. In one embodiment the one-body actuation assembly 13 has the needle protector 14 in which the lancet needle 15 is embedded. The needle protector 14 is easily twisted off of the lancet body 17 before using the device to exposure the lancet needle 15. The lancet needle 15 is mounted on the pedestal 16 attached on the lancet body 17 that is again connected to the divider 18

In one embodiment, on each side of the front surface of the divider 18, attached are a pair of repelling springs 19. In one embodiment, on the posterior surface of the divider 18, attached is the actuation spring 20 that is again connected to the actuator 21. The housing 12 of FIG. 12 is composed of the main case 23 and the front barrel 22. The main case 23 has the posterior opening 30 through which the actuation assembly 13 is inserted into the housing 12 for assembling. The flanges 32 of the end unit 33 engage the open recess 45 of the housing 12, and simply the assembling process is completed.

In one embodiment, on its outer walls, the main case 23 has side walls 40 that have convex configuration outside, and reciprocally concave inside. The pair of the concave groove on the inner side walls 40 engage the convex lateral edges of the butterfly-shape diver 46. This engagement prevents side motion and shaking of the moving lancet body 17 and the lancet 15 when the lancing device is fired, thus helping to minimize puncture pain.

In one embodiment the divider 18 along with the lancet body 17 moves smoothly forward as the convex lateral edge of the butterfly-shape divider 46 slides along the longitudinal grooves of the concave inner side walls 40.

Most of the operating mechanism is described above. In one embodiment the patient prepares the skin where the puncturing by the lancet needle is to be done by sterilizing the area by alcohol swab. In one embodiment the needle protector 14 is twisted off the lancet body 17. In one embodiment the lancing device 11, 60 is held with fingers is placed on the desirable area of the skin.

It will be understood that the foregoing is only illustrative of the principles of the invention, and that various modifications, alterations, and combinations can be made by those skilled in the art without departing from the scope and spirit of the invention. Accordingly, it is not intended that the invention be limited, except as by the appended claims.

What is claimed is:

1. A lancing apparatus configured to reduce pain in taking body fluid samples from the skin, the lancing apparatus comprising:
a housing with an inner space; and
an actuation assembly comprising a lancet needle, a divider and an actuator, the lancet needle having a retracted needle configuration and an extended configuration, the retracted needle configuration enclosing the lancet needle within the housing, the extended configuration at least partially extending the lancet needle outside the housing, the lancet needle operably connected to and extending distally from the divider, the divider operably connected to and distal to the actuator, the divider having a divider front surface and a divider back surface, the divider axially moveable within the inner space, the actuator configured for actuating the lancet needle from the retracted needle configuration to the extended configuration, wherein at least one of the divider front surface and the divider back surface is fluidly connected to a substantially sealed chamber within the inner space, the substantially sealed chamber configured for using a relative differential pressure to actuate the lancet needle from the extended configuration to the retracted needle configuration.

2. The lancing apparatus of claim 1, wherein the substantially sealed chamber is a posterior chamber with a relatively lower differential pressure.

3. The lancing apparatus of claim 2, the posterior chamber fluidly connected to the divider back surface.

4. The lancing apparatus of claim 1, wherein the substantially sealed chamber is an anterior chamber with a relatively higher differential pressure.

5. The lancing apparatus of claim 4, the anterior chamber fluidly connected to the divider front surface.

6. The lancing apparatus of claim 1, wherein the substantially sealed chamber is a posterior chamber fluidly connected to the divider back surface, the posterior chamber with a relatively lower differential pressure than an anterior chamber fluidly connected to the divider front surface.

7. The lancing apparatus of claim 1, further comprising at least one repelling spring operably connected to the divider front surface.

8. The lancing apparatus of claim 1, further comprising at least one actuation spring operably connected to the divider back surface.

9. A differential pressure actuated lancing apparatus, the lancing apparatus comprising:
a housing with an inner space, the inner space having a posterior chamber and an anterior chamber; and
an actuation assembly comprising a lancet needle and a divider, the lancet needle having a retracted needle configuration and an extended configuration, the retracted needle configuration enclosing the lancet needle within the housing, the extended configuration at least partially extending the lancet needle outside the housing, the lancet needle operably connected to and extending distally from the divider, the divider having a divider front surface in fluid communication with the anterior chamber and a divider back surface in fluid communication with the posterior chamber, the divider axially moveable within the inner space, wherein the anterior chamber has a relatively higher differential pressure than the posterior chamber in the extended configuration, the differential pressure actuating the divider proximally toward the anterior chamber.

10. The differential pressure actuated lancing apparatus of claim 9, further comprising at least one repelling spring operably connected to the divider front surface to assist in retracting the lancet needle in to the housing.

11. The differential pressure actuated lancing apparatus of claim 9, further comprising at least one actuation spring operably connected to the divider back surface.

12. The differential pressure actuated lancing apparatus of claim 11, the at least one actuation spring configured to assist in exposing the lancet needle out of the housing.

13. The differential pressure actuated lancing apparatus of claim 11, the at least one actuation spring configured to assist in retracting the lancet needle in to the housing.

14. The differential pressure actuated lancing apparatus of claim 9, further comprising an actuator mechanically connected to the divider for actuating the divider from a locked retracted needle configuration to the extended configuration.

15. A method of actuating a lancing apparatus, comprising:
actuating an actuator operably connected to a lancet needle and a divider, the actuator actuating the lancet needle from a retracted needle configuration to an extended configuration, the retracted needle configuration enclosing the lancet needle within a housing, the extended configuration at least partially extending the lancet needle outside the housing, the divider operably connected to and distal to the actuator, the divider having a divider front surface and a divider back surface, the divider axially moveable within the housing;
retracting the lancet needle from the extended configuration to the retracted needle configuration using a relatively lower differential pressure in a posterior chamber fluidly connected to the divider back surface relative to an anterior chamber fluidly connected to the divider front surface.

16. The method of actuating a lancing apparatus of claim 15, further comprising advancing the lancet needle out of the housing with at least one actuation spring operably connected to the divider back surface.

17. The method of actuating a lancing apparatus of claim 15, further comprising retracting the lancet needle in to the housing with at least one actuation spring operably connected to the divider back surface.

18. The method of actuating a lancing apparatus of claim 15, further comprising retracting the lancet needle in to the housing with at least one repelling spring operably connected to the divider front surface.
